(19) United States
(12) Patent Application Publication
(10) Pub. No.: US 2009/0042942 A1
(43) Pub. Date: Feb. 12, 2009

(54) Muscarinic Receptor Antagonists

(75) Inventors: Andrew Bailey, Leicestershire (GB); David Donald, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/911,933

(22) PCT Filed: Apr. 18, 2006
(86) PCT No.: PCT/SE2006/000459

§ 371 Date: Apr. 10, 2008

(30) Foreign Application Priority Data
Apr. 20, 2005 (SE) 0500893-3
Dec. 7, 2005 (SE) 0502696-8

(51) Int. Cl. A61K 31/445; C07D 207/04; A61K 31/403; C07D 211/60

(52) U.S. Cl. 514/327; 548/556; 514/424; 546/242

(57) ABSTRACT

The invention provides compounds of formula (I), wherein R¹, R², R³, R⁴, R⁵, n and X are as defined in the specification, a process for their preparation, pharmaceutical compositions containing them, a process for preparing pharmaceutical compositions, their use in therapy and intermediates of use in their preparation.

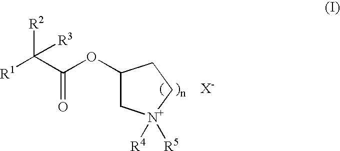

The present invention relates to substituted alkyl esters of cyclic amino alcohols, a process for their preparation, pharmaceutical compositions containing them, a process for preparing pharmaceutical compositions, their use in therapy and intermediates of use in their preparation.

Muscarinic receptors are a G-protein coupled receptor (GPCR) family having five family members M₁, M₂, M₃, M₄ and M₅. Of the five muscarinic subtypes, three (M₁, M₂ and M₃) are known to exert physiological effects on human lung tissue.

Parasympathetic nerves are the main pathway for reflex bronchoconstriction in human airways and mediate airway tone by releasing acetylcholine onto muscarinic receptors. Airway tone is increased in patients with respiratory disorders such as asthma and chronic obstructive pulmonary disease (COPD), and for this reason muscarinic receptor anatgonists have been developed for use in treating airway diseases. Muscarinic receptor antagonists, often called anticholinergics in clinical practice, have gained widespread acceptance as a first-line therapy for individuals with COPD, and their use has been extensively reviewed in the literature (e.g. Lee et al, Current Opinion in Pharmacology 2001, 1, 223-229).

When used to treat respiratory disorders, muscarinic receptor antagonists are typically administered by inhalation. However, when administered by inhalation a significant proportion of the muscarinic receptor antagonist is often absorbed into the systemic circulation resulting in reported side effects such as dry mouth. Additionally, the majority of muscarinic antagonists have a relatively short duration of action requiring that they be administered several times a day. Such a multiple-daily dosing regime is not only inconvenient to the patient but also creates a significant risk of inadequate treatment due to patient non-compliance associated with the frequent repeat dosing schedule.

There therefore remains a need for novel compounds that are capable of blocking muscarinic receptors. In particular, a need exists for new muscarinic antagonists that have high potency and reduced systemic side effects when administered by inhalation. Moreover, a need exists for new muscarinic antagonists that exhibit a long duration of action when dosed by inhalation, and which are amenable to either once or twice daily dosing.

Hydroxyarylcycloalkylcarboxylic esters of cyclic amino alcohols having muscarinic antagonist properties are known, for example from EP 1302458.

In accordance with the present invention, there is provided a compound of formula (I):

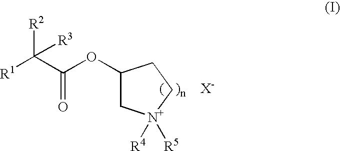

wherein:
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁵ represents hydrogen or C₁₋₆ alkyl;
n is 1 or 2;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
and X represents a pharmaceutically acceptable anion of a mono or polyvalent acid.

The compounds of formula (I) comprise an equivalent of an anion X associated with the positive charge on the quaternary nitrogen atom. The anion X may be any pharmaceutically acceptable anion of a mono or polyvalent (e.g. bivalent) acid. In an embodiment of the invention X may be an anion of a mineral acid, for example chloride, bromide, iodide, sulfate, nitrate or phosphate; or an anion of a suitable organic acid, for example acetate, maleate, fumarate, citrate, oxalate, succinate, tartrate, methanesulphonate or p-toluenesulphonate.

It will be understood that certain compounds of the present invention may exist in solvated, for example hydrated, as well as unsolvated forms. It is to be understood that the present invention encompasses all such solvated forms. Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

In the context of the present specification the term ‘Heteroaromatic’ denotes aromatic rings comprising at least one heteroatom selected from the group consisting of nitrogen, oxygen and sulfur. Examples of 5-6 membered heteroaromatic rings according to the present invention include thienyl, furanyl, pyridinyl, pyrimidinyl, pyrazinyl, pyridazinyl, thiazolyl, oxazolyl, oxadiazolyl, imidazolyl, isoxazolyl, isothiazolyl, pyrazolyl and triazolyl. The term ‘Aliphatic heterocyclic ring’ denotes non-aromatic rings comprising at least one heteroatom selected from the group consisting of nitrogen, oxygen and sulfur. Examples of 4-8 membered aliphatic heterocyclic rings according to the present invention include pyrrolidinyl, piperidinyl, piperazinyl, morpholinyl, homopiperazinyl, homopiperidinyl and azetidinyl.

Unless otherwise stated, in the context of the present specification alkyl groups and moieties may be straight or branched chain and include, for example, methyl, ethyl, n-propyl, iso-propyl or tert-butyl. Cycloalkyl groups are monocyclic, for example cyclopentyl or cyclohexyl. Halogen is for example, fluorine, chlorine or bromine.

In the context of the present specification, where it is stated that a group may be optionally substituted with one or more substituents the group may be unsubstituted or substituted; when substituted the group will generally be substituted with one, two or three substituents.

In an embodiment of the invention, R¹ represents phenyl, which phenyl may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R¹ represents phenyl which may be optionally substituted by one or more substituents independently selected from halogen, hydroxyl, NH₂, NH(C₁₋₄ alkyl) and N(C₁₋₄ alkyl)₂.

In an embodiment of the invention, R² represents cyclopentyl which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂. In a further aspect of this embodiment, R² represents cyclopentyl which may be optionally substituted by one or more substituents independently selected from halogen, hydroxyl, NH₂, NH(C₁₋₄ alkyl) and N(C₁₋₄ alkyl)₂.

In an embodiment of the invention, R³ represents methyl, ethyl or n-propyl. In a further aspect of this embodiment, R³ represents methyl.

In an embodiment of the invention, R⁴ represents hydrogen, methyl, ethyl or n-propyl. In a further aspect of this embodiment, R⁴ represents methyl.

In an embodiment of the invention, R⁵ represents methyl, ethyl or n-propyl. In a further aspect of this embodiment, R⁵ represents methyl.

In an embodiment of the invention, R⁴ and R⁵ each independently represent methyl or ethyl.

In an embodiment of the invention, n is 1.

In an embodiment of the invention, n is 2.

In an embodiment of the invention, X represents chloride, bromide, iodide or fumarate. In a further aspect of this embodiment, X represents bromide or iodide.

In an embodiment of the invention, R¹ represents phenyl which may be optionally substituted by one or more substituents independently selected from halogen, hydroxyl, NH₂, NH(C₁₋₄ alkyl) and N(C₁₋₄ alkyl)₂; R² represents cyclopentyl which may be optionally substituted by one or more substituents independently selected from halogen, hydroxyl, NH₂, NH(C₁₋₄ alkyl) and N(C₁₋₄ alkyl)₂; R³ represents methyl; R⁴ and R⁵ each independently represent methyl or ethyl; n is 1 or 2; and X represents a pharmaceutically acceptable anion of a mono or polyvalent acid. In a further aspect of this embodiment, X represents chloride, bromide, iodide or fumarate.

The compounds of the invention have at least two chiral centers. The position of the two chiral centers is indicated by an asterix (*) in the representation of formula (I) below. One of the chiral centers is located at the carbon atom to which each of R¹, R² and R³ are bonded (the 2′ position), whilst the other chiral center is located at the carbon atom attaching the nitrogen-containing ring to the carboxyl moiety (the 3 position). As the compounds have two chiral centers, for each compound there are four possible steroisomers (3R,2′R) (3S,2′S), (3R,2′S) and (3S,2′R), in two enantiomeric pairs [(3R,2′R)/(3S,2′S) and (3R,2′S)/(3S,2′R)]. The present invention encompasses all optical isomers of the compounds of formula (I) and mixtures thereof including racemates.

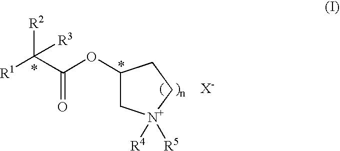

In an embodiment of the invention, the chiral center located at the 3 position has an R configuration. In another embodiment of the invention, the chiral center located at the 3 position has an S configuration. In a further embodiment of the invention, the chiral center located at the 2′ position has an R configuration. In a still further embodiment of the invention, the chiral center located at the 2′ position has an S configuration.

In an embodiment of the invention, the compound of formula (I) has a (3R,2′R) configuration. In another embodiment of the invention, the compound of formula (I) has a (3S,2′S) configuration. In a further embodiment of the invention, the compound of formula (I) has a (3R,2′S) configuration. In a still further embodiment of the invention, the compound of formula (I) has a (3S,2′R) configuration.

In a further embodiment, the present invention provides an optically pure compound of formula (I). In the context of the present specification, the term optically pure is defined in terms of enantiomeric excess (e.e.), which is calculated from the ratio of the difference between the amounts of the respective enantiomers present and the sum of these amounts, expressed as a percentage. To illustrate, a preparation containing 95% of one enantiomer and 5% of another enantiomer has an enantiomeric excess (e.e.) of 90% [i.e. (95−5)/(95+5)×100]. Optically pure compounds according to the present invention have an e.e. of at least 90%. In an embodiment of the invention, optically pure compounds have an e.e. of at least 95%. In a further embodiment of the invention, optically pure compounds have an e.e. of at least 98%. Where the compound has a diastereoisomer, optically pure compounds have an e.e. of at least 90% and a diastereomeric excess (d.e.) of at least 90% [diastereomeric excess being defined by analogy to enantiomeric excess]. In an embodiment of the invention, optically pure compounds have an e.e. of at least 95% and a d.e. of at least 95%. In a further embodiment of the invention, optically pure compounds have an e.e. of at least 98% and a d.e. of at least 98%.

In an embodiment of the invention, there is provided an optically pure compound of formula (I) having a (3R,2′R) configuration.

In an embodiment of the invention, there is provided an optically pure compound of formula (I) having a (3S,2′S) configuration.

In an embodiment of the invention, there is provided an optically pure compound of formula (I) having a (3R,2′S) configuration.

In an embodiment of the invention, there is provided an optically pure compound of formula (I) having a (3S,2′R) configuration.

In one aspect the present invention provides a compound of formula (IB):

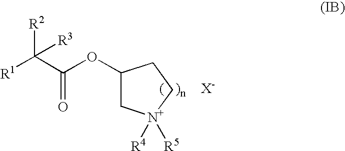

wherein
R¹ represents phenyl, which phenyl may be optionally substituted with halogen, hydroxyl, C₁₋₆ alkyl or C₁₋₆ alkoxy; or R¹ represents thienyl or furanyl, which thienyl or furanyl may be optionally substituted with halogen or C₁₋₆ alkyl;
R² represents a C₃₋₈ cycloalkyl ring, which cycloalkyl ring may be optionally substituted with halogen;
R³ represents C₁₋₆ alkyl;
R⁴ represents hydrogen or C₁₋₆ alkyl;
R⁵ represents C₁₋₆ alkyl;
n is 1 or 2;
and X represents a pharmaceutically acceptable anion of a mono or polyvalent acid.

In an embodiment of the invention, in formula (IB) R¹ represents phenyl.

In an embodiment of the invention, in formula (IB) R² represents a C₃₋₈ cycloalkyl ring, which cycloalkyl ring may be optionally substituted with halogen. When the cycloalkyl ring is substituted with halogen it may be substituted with more than one halogen substituent, and each carbon atom in the cycloalkyl ring may optionally carry one or two halogen substituents.

In an embodiment of the invention, in formula (IB) R² represents an unsubstituted C₃₋₈ cycloalkyl ring. In a further aspect of this embodiment, R² represents unsubstituted cyclopentyl.

In an embodiment of the invention, in formula (IB) R¹ represents cyclopentyl, which cyclopentyl may be optionally substituted with fluorine.

In an embodiment of the invention, in formula (IB) R³ represents methyl, ethyl or n-propyl. In a further aspect of this embodiment, R³ represents methyl.

In an embodiment of the invention, in formula (IB) R⁴ represents hydrogen, methyl, ethyl or n-propyl. In a further aspect of this embodiment, R⁴ represents methyl.

In an embodiment of the invention, in formula (IB) R⁵ represents methyl, ethyl or n-propyl. In a further aspect of this embodiment, R⁵ represents methyl.

In an embodiment of the invention, in formula (IB) n is 1.

In an embodiment of the invention, in formula (IB) R⁴ and R⁵ each independently represent methyl or ethyl, and n is 1.

In an embodiment of the invention, in formula (IB) X represents chloride, bromide, iodide or fumarate. In a further aspect of this embodiment, X represents bromide or iodide.

In an embodiment of the invention, in formula (IB) R¹ represents phenyl; R² represents cyclopentyl; R³ represents methyl; R⁴ and R⁵ each independently represent methyl or ethyl; n is 1 and X represents a pharmaceutically acceptable anion of a mono or polyvalent acid. In a further aspect of this embodiment, X represents chloride, bromide, iodide or fumarate.

In a further aspect, the present invention provides a compound of formula (IC):

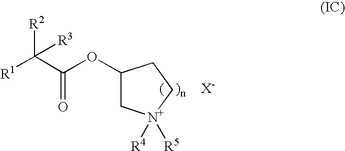

wherein:
R¹ represents phenyl, thienyl or furanyl;
R² represents a C₃₋₈ cycloalkyl ring, which cycloalkyl ring can be optionally substituted with halogen;
R³ represents C₁₋₆ alkyl;
R⁴ represents hydrogen or C₁₋₆ alkyl;
R⁵ represents C₁₋₆ alkyl;
n is 1 or 2;
and X⁻ represents a pharmaceutically acceptable anion of a mono or polyvalent acid.

In an embodiment of the invention, in formula (IC) R¹ represents phenyl.

In an embodiment of the invention, in formula (IC) R² represents a C₃₋₈ cycloalkyl ring, which cycloalkyl ring can be optionally substituted with halogen. When the cycloalkyl ring is substituted by halogen it may be substituted with more than one halogen substituent, and each carbon atom in the cycloalkyl ring may optionally carry one or two halogen substituents.

In an embodiment of the invention, in formula (IC) R¹ represents an unsubstituted C₃₋₈ cycloalkyl ring. In a further aspect of this embodiment, R² represents unsubstituted cyclopentyl.

In an embodiment of the invention, in formula (IC) R² represents cyclopentyl, which cyclopentyl can be optionally substituted with fluorine.

In an embodiment of the invention, in formula (IC) R³ represents methyl, ethyl or n-propyl. In a further aspect of this embodiment, R³ represents methyl.

In an embodiment of the invention, in formula (IC) R⁴ represents hydrogen, methyl, ethyl or n-propyl. In a further aspect of this embodiment, R⁴ represents methyl.

In an embodiment of the invention, in formula (IC) R⁵ represents methyl, ethyl or n-propyl. In a further aspect of this embodiment, R⁵ represents methyl.

In an embodiment of the invention, in formula (IC) n is 1.

In an embodiment of the invention, in formula (IC) R⁴ and R⁵ each independently represent methyl or ethyl; and n is 1.

In an embodiment of the invention, in formula (IC) X represents chloride, bromide or fumarate. In a further aspect of this embodiment, X⁻ represents bromide.

In an embodiment of the invention, in formula (IC) R¹ represents phenyl; R² represents cyclopentyl; R³ represents methyl; R⁴ and R⁵ each independently represent methyl or ethyl; n is 1 and X⁻ represents a pharmaceutically acceptable anion of a mono or polyvalent acid. In a further aspect of this embodiment, XC represents chloride, bromide or fumarate.

In an embodiment of the invention, the compound of formula (I) is selected from:
(3S)-3-{[(2R)-2-Cyclopentyl-2-phenylpropanoyl]oxy}-1,1-dimethylpyrrolidinium X,
(3R)-3-{[(2R)-2-Cyclopentyl-2-phenylpropanoyl]oxy}-1,1-dimethylpyrrolidinium X,
(3S)-3-{[(2S)-2-Cyclopentyl-2-phenylpropanoyl]oxy}-1,1-dimethylpyrrolidinium X,
(3R)-3-{[(2S)-2-Cyclopentyl-2-phenylpropanoyl]oxy}-1,1-dimethylpyrrolidinium X,
(3R)-3-{[(2S)-2-Cyclopentyl-2-phenylpropanoyl]oxy}-1,1-dimethylpiperidinium X, and
(3S)-3-{[(2S)-2-Cyclopentyl-2-phenylpropanoyl]oxy}-1,1-dimethylpiperidinium X,
   wherein X represents a pharmaceutically acceptable anion of a mono or polyvalent acid. Pharmaceutically acceptable anions according to this embodiment include chloride, bromide, iodide and fumarate.

In a further aspect, the present invention provides a process for the preparation of compounds of formula (I), which comprises reacting a compound of formula (IV) wherein R¹, R² and R³ are as defined in formula (I)

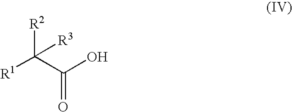

or a C₁₋₆alkyl ester, acid anhydride or acid halide thereof, with a compound of formula (V), wherein R⁴ and n are as defined in formula (I)

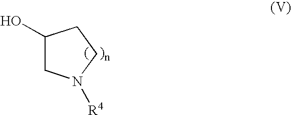

to yield a compound of formula (II)

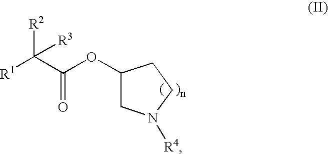

and subsequently reacting (II) with a compound of formula R⁵—Y (III), wherein Y is a leaving group (e.g. halogen) and R⁵ is as defined in formula (I), and optionally carrying out one or more of the following:
converting the compound to a further compound of formula (I),
forming a pharmaceutically acceptable salt with an anion of a mono or polyvalent acid.

The reaction of compounds (IV) and (V) may be conveniently conducted in the presence of a suitable solvent such as toluene or dichloromethane at a temperature in the range of 0 to 100° C. In one embodiment of the invention, compound (IV) may conveniently take the form of an acid halide (e.g. chloride) as may be prepared by reacting the acid with a suitable reagent (e.g. thionyl chloride or oxalyl chloride) in a suitable solvent such as dichloromethane or toluene, at a temperature in the range of 0 to 100° C.

The reaction of compounds (II) and (III) may be conveniently conducted in the presence of a suitable solvent such as dichloromethane or acetonitrile at a temperature in the range of 0 to 100° C.

It will be appreciated by those skilled in the art that in the processes of the present invention certain functional groups such as hydroxyl, carboxyl or amino groups in the starting reagents or intermediate compounds may need to be protected by protecting groups. Thus, the preparation of the compounds of formula (I) may involve at a certain stage the removal of one or more protecting groups. The protection and deprotection of functional groups is described in ‘Protective Groups in Organic Synthesis’, 2nd edition, T. W. Greene and P. G. M. Wuts, Wiley-Interscience (1991) and ‘Protecting Groups’, P. J. Kocienski, Georg Thieme Verlag (1994).

Compounds of formula (IV) may be prepared using methods described, or analogous to those described, in the art. For example, the preparation of α-cycloalkyl-α-phenylpropionic acids is described in Zhumal Obshchei Khimii, (1964), 34 (5), 1618-1621.

Compounds of formula (V) can be obtained commercially or can be prepared using methods described in the literature, e.g. see Chemische Berichte/Recueil (1997), 130(3), 385-397, and Journal of Medicinal & Pharmaceutical Chemistry (1959), 1, 73-94.

Optically pure compounds of formula (I) may, for example, be prepared by reacting an optically pure compound of formula (IV) with an optically pure compound of formula (V), to give an ester of formula (II), and subsequently reacting (II) with a compound of formula (III). Optically pure compounds of formula (V) can be obtained commercially or can be prepared using methods described in the literature.

Optically pure compounds of formula (IV) may be prepared by reacting the corresponding racemic acid, or C₁₋₆alkyl ester, acid anhydride or acid halide thereof, with a suitable auxiliary compound, (e.g. (R)-4-benzyl-2-oxazolidinone), separating the resulting mixture of diastereomeric esters (e.g. by chromatography), and subsequently removing the auxiliary compound to yield an enantiomerically pure acid of formula (IV).

Optically pure compounds of formula (IV) have not been prepared previously. Accordingly, the present invention further provides an optically pure compound of formula (IV)

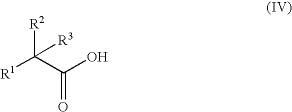

wherein,
R¹ phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁶, R¹³, R⁵, R⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
or a C₁₋₆ alkyl ester, acid anhydride or acid halide thereof.

In an embodiment of the invention, there is provided an optically pure compound of formula (IV) wherein R¹ represents phenyl, which phenyl may be optionally substituted with halogen, hydroxyl, C₁₋₆ allyl or C₁₋₆ alkoxy; or R¹ represents thienyl or furanyl, which thienyl or furanyl may be optionally substituted with halogen or C₁₋₆ alkyl; R² represents a C₃₋₈ cycloalkyl ring, which cycloalkyl ring may be optionally substituted with halogen; R³ represents C₁₋₆ alkyl; or a C₁₋₆alkyl ester, acid anhydride or acid halide thereof.

The optically pure compounds of formula (IV) have an e.e. of at least 90%. In an embodiment of the invention, optically pure compounds of formula (IV) have an e.e. of at least 95%. In a further embodiment of the invention, optically pure compounds of formula (IV) have an e.e. of at least 98%.

Examples of optically pure compounds of formula (IV) according to the present invention include:
(2R)-2-Cyclopentyl-2-phenylpropanoic acid, and
(2S-2-Cyclopentyl-2-phenylpropanoic acid.

Compounds of formula (II) have not been prepared previously. Moreover, these non-quaternised compounds also display activity as anticholinergic agents and are of interest for use in treating conditions of the urinary tract, such as overactive bladder. Accordingly, the present invention further provides a compound of formula (II)

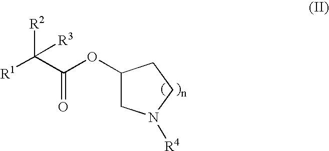

wherein,
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
n is 1 or 2;
R⁶, R¹³, R¹⁵, R¹⁷, R¹⁹, R²³, R²⁴, R³², R³⁴ and R³⁵ seach independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
or any of R⁷ and R⁸, R⁹ and R¹⁰, R¹¹ and R¹², W¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl.

In an embodiment of the invention, there is provided a compound of formula (II) wherein R¹ represents phenyl, which phenyl may be optionally substituted with halogen, hydroxyl, C₁₋₆ alkyl or C₁₋₆ alkoxy; or R¹ represents thienyl or furanyl, which thienyl or furanyl may be optionally substituted with halogen or C₁₋₆ alkyl; R² represents a C₃₋₈ cycloalkyl ring, which cycloalkyl ring may be optionally substituted with halogen; R³ represents C₁₋₆ alkyl; R⁴ represents hydrogen or C₁₋₆ alkyl; and n is 1 or 2.

Compounds of formula (II) according to the present invention include:
(3S)-1-Methylpyrrolidin-3-yl (2R)-2-cyclopentyl-2-phenylpropanoate,
(3R)-1-Methylpyrrolidin-3-yl (2R)-2-cyclopentyl-2-phenylpropanoate,
(3S)-1-Methylpyrrolidin-3-yl (2S)-2-cyclopentyl-2-phenylpropanoate,
(3R)-1-Methylpyrrolidin-3-yl (2S)-2-cyclopentyl-2-phenylpropanoate,
(3R)-1-Methylpiperidin-3-yl (2S)-2-cyclopentyl-2-phenylpropanoate, and
(3S)-1-Methylpiperidin-3-yl (2S)-2-cyclopentyl-2-phenylpropanoate.

The compounds of the invention have activity as pharmaceuticals, in particular as anticholinergic agents including muscarinic receptor (M1, M2, and M3) antagonists, in particular M3 antagonists. Diseases and conditions which may be treated with the compounds include:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus;
2. bone and joints: arthritides associated with or including osteoarthritis/osteoarthrosis, both primary and secondary to, for example, congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthitides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritis, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia greata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, flmgal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and mCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfasion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leulcaemias) and lymphoproliferative systems, such as Hodgkin's and non-Hodgkin's lymphoma; including the prevention and treatment of metastatic disease and tumour recurrences, and paraneoplastic syndromes; and,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

Accordingly, the present invention further provides a compound of formula (I), as hereinbefore defined for use in therapy.

In another aspect, the invention provides the use of a compound of formula (I), as hereinbefore defined, in the manufacture of a medicament for use in therapy.

In the context of the present specification, the term “therapy” also includes “prophylaxis” unless there are specific indications to the contrary. The terms “therapeutic” and “therapeutically” should be construed accordingly.

A further aspect of the invention provides a method of treating a disease state in a mammal suffering from, or at risk of, said disease, which comprises administering to a mammal in need of such treatment a therapeutically effective amount of a compound of formula (I) as hereinbefore defined.

The present invention also provides the use of a compound of formula (I) as hereinbefore defined, in the manufacture of a medicament for use in the treatment of chronic obstructive pulmonary disease (COPD) (such as irreversible COPD).

The present invention also provides the use of a compound of formula (I) as hereinbefore defined, in the manufacture of a medicament for use in the treatment of asthma.

The present invention further provides a method of treating chronic obstructive pulmonary disease (COPD) (such as irreversible COPD), in a warm-blooded animal, such as man, which comprises administering to a mammal in need of such treatment an effective amount of a compound of formula (I) as hereinbefore defined.

The present invention further provides a method of treating asthma in a warm-blooded animal, such as man, which comprises administering to a mammal in need of such treatment an effective amount of a compound of formula (I) as hereinbefore defined.

In order to use a compound of the invention for the therapeutic treatment of a warm-blooded animal, such as man, said ingredient is normally formulated in accordance with standard pharmaceutical practice as a pharmaceutical composition.

Therefore in another aspect the present invention provides a pharmaceutical composition that comprises a compound of the invention as hereinbefore defined and a pharmaceutically acceptable adjuvant, diluent or carrier. In a further aspect the present invention provides a process for the preparation of said composition, which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), oral, rectal or parenteral administration. For these purposes the compounds of this invention may be formulated by means known in the art into the form of, for example, aerosols, dry powder formulations, tablets, capsules, syrups, powders, granules, aqueous or oily solutions or suspensions, (lipid) emulsions, dispersible powders, suppositories, ointments, creams, drops and sterile injectable aqueous or oily solutions or suspensions.

A suitable pharmaceutical composition of this invention is one suitable for oral administration in unit dosage form, for example a tablet or capsule, which contains between 0.1 mg and Ig of active ingredient.

In another aspect a pharmaceutical composition of the invention is one suitable for intravenous, subcutaneous or intramuscular injection. Each patient may receive, for example, an intravenous, subcutaneous or intramuscular dose of 0.01 mgkg⁻¹ to 100 mgkg⁻¹ of the compound, for example in the range of 0.1 mgkg⁻¹ to 20 mgkg⁻¹ of this invention, the composition being administered 1 to 4 times per day. The intravenous, subcutaneous and intramuscular dose may be given by means of a bolus injection. Alternatively the intravenous dose may be given by continuous infusion over a period of time. Alternatively each patient will receive a daily oral dose, which is approximately equivalent to the daily parenteral dose, the composition being administered 1 to 4 times per day

Another suitable pharmaceutical composition of this invention is one suitable for inhaled administration, inhalation being a particularly useful method for administering the compounds of the invention when treating respiratory diseases such as chronic obstructive pulmonary disease (COPD) or asthma. When administered by inhalation the compounds of formula (I) may be used effectively at doses in the μg range, for example 0.1 to 500 μg, 0.1 to 50 μg, 0.1 to 40 μg, 0.1 to 30 μg, 0.1 to 20 μg, 0.1 to 10 μg, 5 to 10 μg, 5 to 50 μg, 5 to 40 μg, 5 to 30 μg, 5 to 20 μg, 5 to 10 μg, 10 to 50 μg, 10 to 40 μg, 10 to 30 μg, or 10 to 20 μg of active ingredient.

In an embodiment of the invention, there is provided a pharmaceutical composition comprising a compound of the invention as hereinbefore defined, in association with a pharmaceutically acceptable adjuvant, diluent or carrier, which is formulated for inhaled administration.

When administered by inhalation, metered dose inhaler devices may be used to administer the active ingredient, dispersed in a suitable propellant and with or without additional excipients such as ethanol, surfactants, lubricants or stabilising agents. Suitable propellants include hydrocarbon, chlorofluorocarbon and hydrofluoroalkane (e.g. heptafluoroalkane) propellants, or mixtures of any such propellants. Preferred propellants are P134a and P227, each of which may be used alone or in combination with other propellants and/or surfactant and/or other excipients. Nebulised aqueous suspensions or, preferably, solutions may also be employed, with or without a suitable pH and/or tonicity adjustment, either as a unit-dose or multi-dose formulations.

Dry powder inhalers may be used to administer the active ingredient, alone or in combination with a pharmaceutically acceptable carrier, in the later case either as a finely divided powder or as an ordered mixture. The dry powder inhaler may be single dose or multi-dose and may utilise a dry powder or a powder-containing capsule.

Metered dose inhaler, nebuliser and dry powder inhaler devices are well known and a variety of such devices are available.

The invention further relates to combination therapies wherein a compound of the invention or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention with an inhibitor of matrix metalloprotease (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention and a receptor antagonist for leukotrienes (LT) B4, LTC4, LTD4, and LTE4 selected from the group consisting of the phenothiazin-3-1s such as L-651,392; amidino compounds such as CGS-25019c; benzoxalamines such as ontazolast; benzenecarboximidamides such as BIIL 284/260; and compounds such as zafirlulkast, ablukast, montelukast, pranlukast, verlukast (MK-679), RG-12525, Ro-245913, iralukast (CGP 45715A), and BAY x 7195.

The present invention still further relates to the combination of a compound of the invention and a phosphodiesterase (PDE) inhibitor such as a methylxanthanine including theophylline and aminophylline; a selective PDE isoenzyme inhibitor including a PDE4 inhibitor an inhibitor of the isoform PDE4D, or an inhibitor of PDE5.

The present invention further relates to the combination of a compound of the invention and a histamine type 1 receptor antagonist such as cetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention and a proton pump inhibitor (such as omeprazole) or a gastroprotective histamine type 2 receptor antagonist.

The present invention further relates to the combination of a compound of the invention and an antagonist of the histamine type 4 receptor.

The present invention still further relates to the combination of a compound of the invention and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention still further relates to the combination of a compound of the invention and a beta-adrenoceptor agonist (including beta receptor subtypes 1-4) such as isoprenaline, salbutamol, formoterol, salmeterol, terbutaline, orciprenaline, bitolterol mesylate, pirbuterol, or indacaterol or a chiral enantiomer thereof.

The present invention further relates to the combination of a compound of the invention and a chromone, such as sodium cromoglycate or nedocromil sodium.

The present invention still further relates to the combination of a compound of the invention with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention with an agent that modulates a nuclear hormone receptor such as PPARs.

The present invention still further relates to the combination of a compound of the invention together with an immunoglobulin (Ig) or Ig preparation or an antagonist or antibody modulating Ig function such as anti-IgE (for example omalizumab).

The present invention further relates to the combination of a compound of the invention and another systemic or topically-applied anti-inflammatory agent, such as thalidomide or a derivative thereof, a retinoid, dithranol or calcipotriol.

The present invention still further relates to the combination of a compound of the invention and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention and an agent for the treatment of acute or chronic pain, such as a centrally or peripherally-acting analgesic (for example an opioid or derivative thereof), carbamazepine, phenyloin, sodium valproate, amitryptiline or other anti-depressant agent-s, paracetamol, or a non-steroidal anti-inflammatory agent.

The present invention further relates to the combination of a compound of the invention together with a parenterally or topically-applied (including inhaled) local anaesthetic agent such as lignocaine or a derivative thereof.

A compound of the present invention can also be used in combination with an anti-osteoporosis agent including a hormonal agent such as raloxifene, or a biphosphonate such as alendronate.

The present invention still further relates to the combination of a compound of the invention together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine linase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B1.- or B2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK1 or NK3 receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of P38; (xxv) agent modulating the is function of Toll-like receptors (TLR), (xxvi) agent modulating the activity of purinergic receptors such as P2×7; or (xxvii) inhibitor of transcription factor activation such as NFkB, API, or STATS.

A compound of the invention can also be used in combination with an existing therapeutic agent for the treatment of cancer, for example suitable agents include:

(i) an antiproliferative/antineoplastic drug or a combination thereof, as used in medical oncology, such as an allylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erbb2 antibody trastuzumab, or the anti-erbb1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αᵥβ₃ function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

A combination of particular interest for use in the treatment of respiratory diseases such as asthma, COPD or allergic rhinitis is a combination of a compound of formula (I), as defined herein, with one or more agents selected from the list comprising:
a PDE4 inhibitor including an inhibitor of the isoform PDE4D,
a selective β2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a modulator of chemokine receptor function (such as a CCR1 receptor antagonist),
an inhibitor of p38 kinase function,
a glucocorticoid, such as flunisolide, triamcinolone acetonide, beclomethasone dipropionate, budesonide, fluticasone propionate, ciclesonide or mometasone furoate, and
a non-steroidal glucocorticoid receptor antagonist.

In an embodiment, the present invention provides a pharmaceutical product comprising, in combination, a first active ingredient which is a compound of formula (I) as hereinbefore defined, and a second active ingredient which is a β2. adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol.

In an embodiment, the present invention provides a pharmaceutical product comprising, in combination, a first active ingredient which is a compound of formula (I) as hereinbefore defined, and a second active ingredient which is a glucocorticoid, such as flunisolide, triamcinolone acetonide, beclomethasone dipropionate, budesonide, fluticasone propionate, ciclesonide or mometasone.

In an embodiment, the present invention provides a pharmaceutical product comprising, in combination, a first active ingredient which is a compound of formula (I) as hereinbefore defined, and a second active ingredient which is a non-steroidal glucocorticoid receptor antagonist.

The present invention will now be illustrated with the following non-limiting Examples.

In the examples the NMR spectra were measured on a Varian Unity Inova spectrometer at a proton frequency of either 300 or 400 MHz. The MS spectra were measured on either an Agilent 1100 MSD G1946D spectrometer or a Hewlett Packard HP1100 MSD G1946A spectrometer. Preparative HPLC separations were performed using a Waters Symmetry® or Xterra® column using 0.1% aqueous trifluoroacetic acid: acetonitrile, 0.1% aqueous ammonia: acetonitrile or 0.1% ammonium acetate: acetonitrile as the eluent. SCX and NH₂ resin were obtained from Varian Incorporated.

### EXAMPLE 1

### (3S)-3-{[(2R)-2-Cyclopentyl-2-phenylpropanoyl]oxy}-1,1-dimethylpyrrolidinium iodide

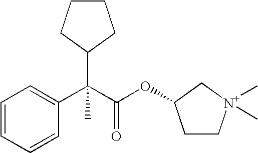

### a) Methyl cyclopentyl(phenyl)acetate

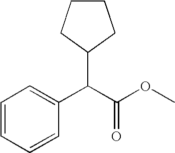

A solution of alpha-phenylcyclopentaneacetic acid (10 g) in methanol (200 mL) was treated with chlorotrimethylsilane (20 mL) and the mixture heated at reflux for 3 hours. The solvent was removed under reduced pressure and the residue was purified by chromatography on a silica column, eluting with 25% diethylether in iso-hexane to yield the sub-titled compound (10.5 g) as an oil.

¹H NMR (400 MHz, CDCl₃) δ 7.35-7.22 (m, 5H), 3.64 (s, 3H), 3.28 (d, 1H), 2.60-2.50 (m, 1H), 1.95-1.85 (m, 1H), 1.70-1.40 (m, 5H), 1.30-1.20 (m, 1H), 1.03-0.97 (m, 1H).

### b) Methyl 2-cyclopentyl-2-phenylpropanoate

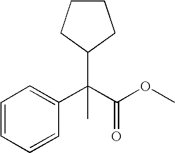

A 1.8 molar solution of lithium diisopropylamide in heptane/tetrahydrofuran/ethylbenzene (40.1 mL) was added to anhydrous tetrahydrofuran (60 mL) stirred at −78° C. under nitrogen. To this mixture was then added, dropwise over 25 minutes, a solution of methyl cyclopentyl(phenyl)acetate (Example 1a), 10.5 g) in anhydrous tetrahydrofuran (30 mL). The reaction mixture was stirred at −78° C. for 15 minutes and then allowed to warm to −30° C. A solution of methyl iodide (13.68 g) in anhydrous tetrahydrofuran (15 mL) was then added over a period of 10 minutes. The mixture was stirred at 0° C. for 1 hour and then partitioned between ethyl acetate and saturated aqueous ammonium chloride solution. The organic layer was separated, dried over anhydrous magnesium sulfate, filtered and the solvent removed under reduced pressure to yield the sub-titled compound (10.4 g) as an oil.

¹H NMR (400 MHz, CDCl₃) δ 7.36-7.19 (m, 5H), 3.64 (s, 3H), 2.83-2.76 (m, 1H), 1.77-1.69 (m, 1H), 1.60-1.30 (m, 9H), 0.95-0.85 (m, 1H).

### c) 2-Cyclopentyl-2-phenyl-propionic acid

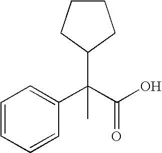

A mixture of Methyl 2-cyclopentyl-2-phenylpropanoate (Example 1b), 10.4 g) in methanol (150 mL) and 2 molar aqueous sodium hydroxide solution (100 mL) was refluxed for 18 hours. The methanol was evaporated under reduced pressure and the residue partitioned between water and diethylether, the aqueous layer was separated and cooled with ice. The aqueous solution was acidified with concentrated hydrochloric acid and the resultant precipitate extracted into ethyl acetate. The organic layer was then washed with brine, separated, dried over anhydrous magnesium sulfate and the solvent removed under reduced pressure to yield the sub-titled compound (7.7 g).

m/e 217 (M−H⁺, 100%)

### d) (4R)-4-Benzyl-3-[(2R)-2-cyclopentyl-2-phenylpropanoyl]-1,3-oxazolidin-2-one

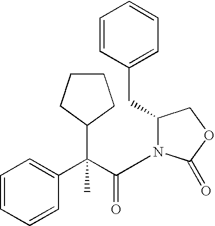

A solution of 2-Cyclopentyl-2-phenyl-propionic acid (Example 1c), 4.93 g) in toluene (100 mL) was treated with thionyl chloride (30 mL) and the resulting mixture heated at 100° C. for 2 hours. The solvent was removed under reduced pressure and the residue azeotroped three times with toluene. The residue was dissolved in anhydrous tetrahydrofuran (20 mL) and added in one portion to a solution of (R)-4-benzyl-2-oxazolidinone (4.00 g) in anhydrous terahydrofuran (100 mL) at −78° C., which had been pre-treated at −78° C. with a 1.6 molar solution of n-butyllithium in hexanes (14.1 mL). The reaction mixture was stirred at −78° C. for 30 minutes and then at room temperature for 1 hour. At the end of this time the mixture was partitioned between ethyl acetate and saturated aqueous ammonium chloride solution, the organic layer was dried over anhydrous magnesium sulfate and evaporated under reduced pressure. Purification was by chromatography on silica gel, eluting with 10% tetrahydrofuran in iso-hexane followed by tritruration of the resultant solid with iso-hexane to yield the sub-titled compound 2.97 g as a white solid.

m/e 378 (M+H⁺, 100%)

¹H NMR (400 MHz, DMSO-D₆) δ 7.33-7.22 (m, 7H), 7.15-7.09 (m, 3H), 4.85-4.81 (m, 1H), 4.24 (t, 1H), 4.09 (q, 1H), 3.04-2.90 (m, 2H), 2.55 (t, 1H), 1.78-1.71 (m, 1H), 1.69 (s, 3H), 1.42-1.20 (m, 6H), 1.15-1.05 (m, 1H).

### e) (2R)-2-Cyclopentyl-2-phenylpropanoic acid

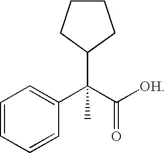

To a solution of (4R)-4-Benzyl-3-[(2R)-2-cyclopentyl-2-phenylpropanoyl]-1,3-oxazolidin-2-one (Example 1d), 2.9 g) in a mixture of tetrahydrofuran (80 mL) and water (20 mL) stirred at 0° C. under nitrogen was added, dropwise a 35 wt. % solution of hydrogen peroxide in water (2.69 mL). The mixture was then treated dropwise with a solution of lithium hydroxide monohydrate (516 mg) in water (7 mL) followed by stirring for 4 hours at room temperature. At the end of this time the reaction mixture was cooled in an ice bath and treated with a solution of sodium metabisulphite (4 g) in water (30 mL). The mixture was evaporated under reduced pressure to remove the tetrahydrofuran and the residue was partitioned between diethyl ether and excess dilute aqueous sodium hydroxide. The aqueous layer was cooled and acidified by dropwise addition of concentrated hydrochloric acid, the resultant precipitate was extracted into ethyl acetate. The combined ethyl acetate extracts were washed with brine, dried over anhydrous magnesium sulfate and evaporated under reduced pressure. The crude product was purified by flash chromatography on a silica column, eluting with 20% ethyl acetate in iso-hexane to yield the sub-titled compound (1.7 g). The enantiomeric excess of the acid was determined by chiral HPLC using a Chiralpak® AD-H column eluting with iso-hexane/2-propanol (9/1) to be greater than 98%.

m/e 217.7 (M−H⁺, 100%)

¹H NMR (400 MHz, CDCl₃) δ 7.42-7.39 (m, 2H), 7.32 (t, 2H), 7.26-7.22 (m, 1H), 2.84-2.80 (m, 1H), 1.80-1.77 (m, 1H), 1.60-1.36 (m, 9H), 1.14-1.09 (m, 1H).

### f) (3S)-1-Methylpyrrolidin-3-yl (2R)-2-cyclopentyl-2-phenylpropanoate

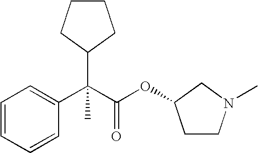

A solution of (2R)-2-Cyclopentyl-2-phenylpropanoic acid (Example 1e), 0.8 g) in toluene (20 mL) was treated with thionyl chloride (5 mL) and the resultant mixture heated at 100° C. for 2 hours. The solvent was removed under reduced pressure and the residue azeotroped three times with toluene yielding 0.87 g of the acid chloride. A solution of the acid chloride (0.43 g) in dichloromethane (7 mL) was treated with S-(+)-3-hydroxy-N-methylpyrrolidine (556 mg) (obtained from Lancaster Synthesis Limited with a quoted e.e. of 99%) and the reaction mixture was heated at 40° C. for 20 hours. The reaction mixture was partitioned between dichloromethane and saturated aqueous sodium bicarbonate solution, the organic layer was dried and evaporated under reduced pressure. The crude product was purified by flash chromatography on a silica column, eluting with 1% triethylamine in ethyl acetate/iso-hexane (1/1) to yield 0.21 g of product.

m/e 302 (M+H⁺, 100%)

### g) (3S)-3-{[(2R)-2-Cyclopentyl-2-phenylpropanoyl]oxy}-1,1-dimethylpyrrolidinium iodide

A solution of (3S)-1-Methylpyrrolidin-3-yl (2R)-2-cyclopentyl-2-phenylpropanoate (Example 1f), 100 mg) in acetonitrile (1 mL) was treated with a solution of methyl iodide (50 mg) in dichloromethane (0.5 mL). The solvents were evaporated under reduced pressure and the residue triturated with diethyl ether to yield the title compound as a white solid (90 mg).

m/e 316 (M⁺, 100%)

¹H NMR (400 MHz, DMSO-D₆) δ 7.35-7.34 (m, 4H), 7.27-7.24 (m, 1H), 5.36 (s, 1H), 3.79 (q, 1H), 3.59-3.54 (m, 2H), 3.51-3.46 (m, 1H), 3.12 (s, 3H), 2.93 (s, 3H), 2.79-2.75 (m, 1H), 2.65-2.60 (m, 1H), 2.05-2.00 (m, 1H), 1.69-1.66 (m, 1H), 1.53-1.33 (m, 9H), 1.12-1.09 (m, 1H).

### EXAMPLE 2

### (3R)-3-{[(2R)-2-Cyclopentyl-2-phenylpropanoyl]oxy}-1,1-dimethylpyrrolidinium iodide

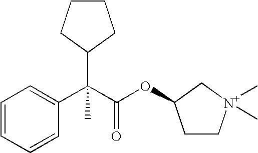

### a) (3R)-1-Methylpyrrolidin-3-yl (2R)-2-cyclopentyl-2-phenylpropanoate

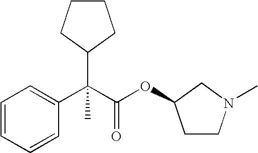

A solution of (2R)-2-Cyclopentyl-2-phenylpropanoic acid (Example 1e), 0.8 g) in toluene (20 mL) was treated with thionyl chloride (5 mL) and the resultant mixture heated at 100° C. for 2 hours. The solvent was removed under reduced pressure and the residue azeotroped three times with toluene yielding 0.87 g of the acid chloride. A solution of the acid chloride (0.43 g) in dichloromethane (7 mL) was treated with (R)-1-methyl-3-hydroxypyrrolidine (556 mg) (obtained from Lancaster Synthesis Limited with a quoted e.e. of 99%) and the reaction mixture was heated at 40° C. for 20 hours. The reaction mixture was partitioned between dichloromethane and saturated aqueous sodium bicarbonate solution, the organic layer was dried and evaporated under reduced pressure. The crude product was purified by flash chromatography on a silica column, eluting with 1% triethylamine in ethyl acetate/iso-hexane (1/1) to yield the sub-titled compound (0.26 g).

m/e 302 (M+H⁺, 100%)

### b) (3R)-3-{[(2R)-2-Cyclopentyl-2-phenylpropanoyl]oxy}-1,1-dimethylpyrrolidinium iodide

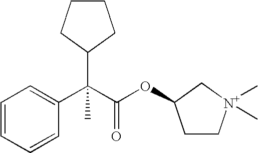

The title compound was prepared from (3R)-1-methylpyrrolidin-3-yl (2R)-2-cyclopentyl-2-phenylpropanoate (Example 2a), 100 mg) using the method of Example 1 Step g) to yield the titled compound (50 mg).

m/e 316 (M⁺, 100%)

¹H NMR (400 MHz, DMSO-D₆) δ 7.35-7.31 (m, 4H), 7.27-7.22 (m, 1H), 5.36 (s, 1H), 3.78 (q, 1H), 3.59-3.54 (m, 2H), 3.51-3.46 (m, 1H), 3.12 (s, 3H), 2.91 (s, 3H), 2.79-2.75 (m, 1H), 2.66-2.60 (m, 1H), 2.06-2.00 (m, 1H), 1.69-1.66 (m, 1H), 1.53-1.33 (m, 9H), 1.12-1.07 (m, 1H).

### EXAMPLE 3

### (3S)-3-{[(2S)-2-Cyclopentyl-2-phenylpropanoyl]oxy}-1,1-dimethylpyrrolidinium iodide

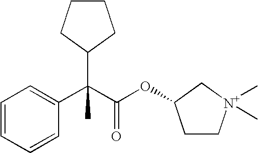

### a) (4R)-4-Benzyl-3-[(2S)-2-cyclopentyl-2-phenylpropanoyl]-1,3-oxazolidin-2-one

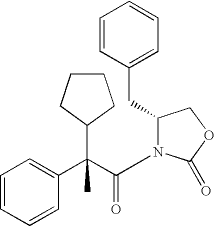

A solution of 2-cyclopentyl-2-phenyl-propionic acid (Example 1c), 4.93 g) in toluene (100 mL) was treated with thionyl chloride (30 mL) and the resultant mixture heated at 100° C. for 2 hours. The solvent was removed under reduced pressure and the residue azeotroped three times with toluene. The residue was dissolved in anhydrous tetrahydrofuran (20 mL) and added in one portion to a solution of (R)-4-benzyl-2-oxazolidinone (4.00 g) in anhydrous terahydrofuran (100 mL) at −78° C. which had been pre-treated at −78° C. with a 1.6 molar solution of n-butyllithium in hexanes (14.1 mL). The reaction mixture was stirred at −78° C. for 30 minutes and then at room temperature for 1 hour. At the end of this time the mixture was partitioned between ethyl acetate and saturated aqueous ammonium chloride solution, the organic layer was dried over anhydrous magnesium sulfate and evaporated under reduced pressure. Purification was by flash chromatography on silica gel eluting with 10% tetrahydrofuran in iso-hexane to yield the sub-titled compound (3.30 g).

¹H NMR (400 MHz, DMSO-D₆) δ 7.32-7.24 (m, 7H), 7.18-7.12 (m, 3H), 4.72-4.70 (m, 1H), 4.20 (t, 1H), 4.01 (q, 1H), 3.20 (q, 1H), 2.81-2.74 (m, 2H), 1.78-1.72 (m, 1H), 1.59 (s, 3H), 1.49-1.17 (m, 7H).

### b) (2S)-2-Cyclopentyl-2-phenylpropanoic acid

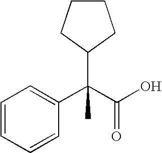

The sub-titled compound was prepared from (4R)-4-Benzyl-3-[(2S)-2-cyclopentyl-2-phenylpropanoyl]-1,3-oxazolidin-2-one (Example 3a), 3.25 g) using the method of Example 1 Step e) to yield the sub-titled compound (1.7 g). The enantiomeric excess of the acid was determined by chiral HPLC using a Chiralpak® AD-H column eluting with iso-hexane/2-propanol (9/1) to be greater than 98%.

m/e 217.7 (M−H⁺, 100%)

¹H NMR (400 MHz, CDCl₃) δ 7.42-7.39 (m, 2H), 7.32 (t, 2H), 7.26-7.22 (m, 1H), 2.84-2.79 (m, 1H), 1.80-1.77 (m, 1H), 1.59-1.38 (m, 9H), 1.14-1.11 (m, 1H).

### c) (3S)-1-Methylpyrrolidin-3-yl (2S)-2-cyclopentyl-2-phenylpropanoate

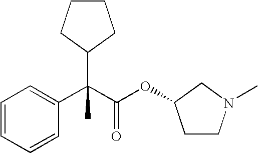

A solution of (2S)-2-Cyclopentyl-2-phenylpropanoic acid (Example 3b), 0.85 g) in toluene (100 mL) was treated with thionyl chloride (15 mL) and the resultant mixture heated at 100° C. for 2 hours. The solvent was removed under reduced pressure and the residue was azeotroped three times with toluene yielding 0.87 g of the acid chloride. A solution of the resulting acid chloride (0.43 g) in dichloromethane (7 mL) was treated with S-(+)-3-hydroxy-N-methylpyrrolidine (556 mg) (obtained from Lancaster Synthesis Limited with a quoted e.e. of 99%) and the reaction mixture was heated at 40° C. for 20 hours. The reaction mixture was partitioned between dichloromethane and saturated aqueous sodium bicarbonate solution, the organic layer was dried and evaporated under reduced pressure. The crude product was purified by flash chromatography on a silica column, eluting with 1% triethylamine in ethyl acetate/iso-hexane (1/1) to yield the sub-titled compound (0.2 g).

m/e 302 (M+H⁺, 100%)

### d) (3S)-3-{[(2S)-2-Cyclopentyl-2-phenylpropanoyl]oxy}-1,1-dimethylpyrrolidinium iodide

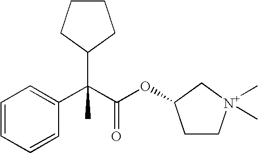

The title compound was prepared from (3S)-1-Methylpyrrolidin-3-yl (2S)-2-cyclopentyl-2-phenylpropanoate (100 mg) using the method of Example 1 Step g) to yield the titled compound (90 mg).

m/e 316 (M⁺, 100%)

¹H NMR (400 MHz, DMSO-D₆) δ 7.35-7.34 (m, 4H), 7.26-7.22 (m, 1H), 5.36 (s, 1H), 3.77 (q, 1H), 3.60-3.43 (m, 3H), 3.12 (s, 3H), 2.91 (s, 3H), 2.79-2.74 (m, 1H), 2.67-2.63 (m, 1H), 2.07-2.04 (m, 1H), 1.69-1.66 (m, 1H), 1.53-1.33 (m, 9H), 1.12-1.07 (m, 1H).

### EXAMPLE 4

### (3R)-3-{[(2S)-2-Cyclopentyl-2-phenylpropanoyl]oxy}-1,1-dimethylpyrrolidinium iodide

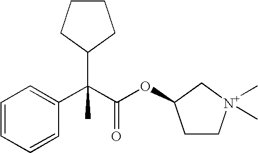

### a) (3R)-1-Methylpyrrolidin-3-yl (2S)-2-cyclopentyl-2-phenylpropanoate

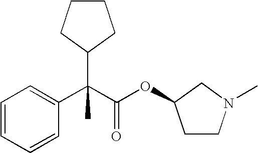

A solution of (2S)-2-Cyclopentyl-2-phenylpropanoic acid (Example 3b), 0.85 g) in toluene (100 mL) was treated with thionyl chloride (15 mL) and the resultant mixture heated at 100° C. for 2 hours. The solvent was removed under reduced pressure and the residue azeotroped three times with toluene yielding 0.87 g of the acid chloride. A solution of the acid chloride (0.43 g) in dichloromethane (7 mL) was treated with (R)-1-methyl-3-hydroxypyrrolidine (556 mg) (obtained from Lancaster Synthesis Limited with a quoted e.e. of 99%) and the reaction mixture was heated at 40° C. for 20 hours. The reaction mixture was partitioned between dichloromethane and saturated aqueous sodium bicarbonate, the organic layer was separated and dried over anhydrous magnesium sulfate and evaporated under reduced pressure. The crude product was purified by flash chromatography on a silica column, eluting with 1% triethylamine in ethyl acetate/iso-hexane (1/1) to yield the sub-titled compound (0.23 g).

m/e 302 (M+H⁺, 100%)

### b) (3R)-3-{[(2S)-2-Cyclopentyl-2-phenylpropanoyl]oxy}-1,1-dimethylpyrrolidinium iodide

The title compound was prepared from (3R)-1-Methylpyrrolidin-3-yl (2S)-2-cyclopentyl-2-phenylpropanoate (Example 4a), 100 mg) using the method of Example 1 Step g) to yield the titled compound (110 mg).

m/e 316 (M⁺, 100%)

¹H NMR (400 MHz, DMSO-D₆) δ 7.35-7.33 (m, 4H), 7.27-7.24 (m, 1H), 5.37 (s, 1H), 3.79 (q, 1H), 3.60-3.55 (m, 2H), 3.51-3.44 (m, 1H), 3.12 (s, 3H), 2.93 (s, 3H), 2.80-2.75 (m, 1H), 2.66-2.61 (m, 1H), 2.04-2.00 (m, 1H), 1.71-1.67 (m, 1H), 1.54-1.33 (m, 9H), 1.12-1.07 (m, 1H).

### EXAMPLE 5

### (3R)-3-{[(2S)-2-Cyclopentyl-2-phenylpropanoyl]oxy}-1,1-dimethylpiperidinium iodide

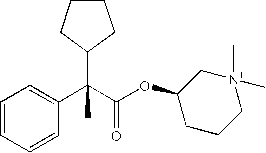

### a) (3R)-1-Methylpiperidin-3-yl (2S)-2-cyclopentyl-2-phenylpropanoate

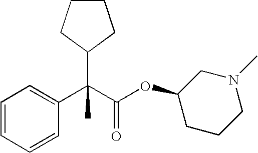

A solution of (2S)-2-Cyclopentyl-2-phenylpropanoic acid (Example 3b), 246 mg) in toluene (20 mL) was treated with thionyl chloride (5 mL) and the resultant mixture heated at 100° C. for 2 hours. The solvent was removed under reduced pressure and the residue azeotroped three times with touluene. The residue was dissolved in dichloromethane (3 mL) and was treated with (R)-3-hydroxy-N-methylpiperidine (130 mg—prepared from commercially available (R)-(+)-3-hydroxypiperidine hydrochloride by the method outlined in European Journal of Medicinal Chemistry (1976), 11(5), 461-6) and the reaction mixture was heated at 40° C. for 18 hours. The reaction mixture was partitioned between dichloromethane and saturated aqueous sodium bicarbonate, the separated organic layer was dried over anhydrous magnesium sulfate and evaporated under reduced pressure. The crude product was purified by flash chromatography on a silica column, eluting with 1% triethylamine in ethyl acetate/iso-hexane (1/1) to yield the sub-titled compound (77 mg).

m/e 316 (M+H⁺, 100%)

### b) (3R)-3-{[(2S)-2-Cyclopentyl-2-phenylpropanoyl]oxy}-1,1-dimethylpiperidinium iodide

The title compound was prepared from (3R)-1-methylpiperidin-3-yl (2S)-2-cyclopentyl-2-phenylpropanoate (77 mg) using the method of Example 1 Step g) to yield the titled compound (85 mg).

m/e 330.3 (M⁺, 100%)

¹H NMR (400 MHz, DMSO-D₆) δ 7.37-7.31 (m, 4H), 7.28-7.24 (m, 1H), 5.15 (s, 1H), 3.56-3.51 (m, 1H), 3.39-3.36 (m, 1H), 3.07 (s, 3H), 2.88 (s, 3H), 2.77-2.73 (m, 1H), 1.82-1.58 (m, 4H), 1.58-1.33 (m, 10H), 1.14-1.07 (m, 1H).

### EXAMPLE 6

### (3S)-3-{[(2S)-2-Cyclopentyl-2-phenylpropanoyl]oxy}-1,1-dimethylpiperidinium iodide

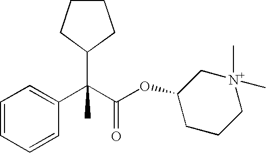

### a) (3S)-1-Methylpiperidin-3-yl (2S)-2-cyclopentyl-2-phenylpropanoate

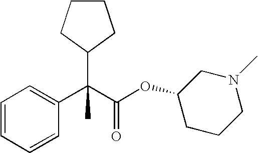

A solution of (2S)-2-Cyclopentyl-2-phenylpropanoic acid (Example 3b), 250 mg) in toluene (20 mL) was treated with thionyl chloride (5 mL) and the resultant mixture heated at 100° C. for 2 hours. The solvent was removed under reduced pressure and the residue azeotroped three times with toluene. The residue was dissolved in dichloromethane (3 mL) and was treated with (S)-3-hydroxy-N-methylpiperidine (250 mg—prepared from commercially available (S)-(−)-3-hydroxypiperidine hydrochloride by the method outlined in European Journal of Medicinal Chemistry (1976), 11(5), 461-6) and the reaction mixture was heated at 40° C. for 18 hours. The reaction mixture was partitioned between dichloromethane and saturated aqueous sodium bicarbonate solution, the separated organic layer was dried over anhydrous magnesium sulfate and evaporated under reduced pressure. The crude product was purified by flash chromatography on a silica column, eluting with 1% triethylamine in ethyl acetate/iso-hexane (1/1) to yield the sub-titled compound (80 mg).

m/e 316 (M+H⁺, 100%)

### b) (3S)-3-{[(2S)-2-Cyclopentyl-2-phenylpropanoyl]oxy}-1,1-dimethylpiperidinium iodide

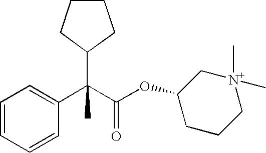

The title compound was prepared from (3S)-1-Methylpiperidin-3-yl (2S)-2-cyclopentyl-2-phenylpropanoate (Example 6a), 80 mg) using the method of Example 1 Step g) to yield the titled compound (90 mg).

m/e 330 (M⁺, 100%)

¹H NMR (400 MHz, DMSO-D₆) δ 7.38-7.33 (m, 4H), 7.28-7.23 (m, 1H), 5.18-5.14 (m, 1H), 3.58-3.53 (m, 1H), 3.35-3.31 (m, 1H), 3.08 (s, 3H), 2.89 (s, 3H), 2.81-2.75 (m, 1H), 1.81-1.75 (m, 2H), 1.75-1.65 (m, 2H), 1.58-1.30 (m, 10H), 1.17-1.11 (m, 1H).

### Pharmacological Analysis

The affinity (pIC₅₀) of compounds to the M₃ receptor was determined by competition binding of [³H]N-methyl scopolamine (NMS) to CHO-KL (Chinese Hamster Ovary) cell membranes expressing the human muscarinic acetylcholine M₃ receptor (M₃-ACh) in a scintillation proximity assay (SPA) format.

Membranes were precoupled to SPA beads 5 μg membrane protein per mg of SPA beads, and then incubated at 2 mg/well with serial dilutions of the compounds of the invention, [³H]NMS at 0.2 nM, half Kd (experimentally determined dissociation constant) and assay buffer (20 mM HEPES pH 7.4 containing 5 mM MgCl₂). The assay was conducted in a final volume of 200 μL, in the presence of 1% (v/v) dimethyl sulphoxide (DMSO). Total binding of [³H]NMS was determined in the absence of competing compound and non-specific binding of [³H]NMS was determined in the presence of 1 μM atropine. The plates were incubated for 16 hours at room temperature and then read on Wallac Microbeta™ using a normalised ³H protocol. The pIC₅₀, defined as the negative logarithm of the concentration of compound required for 50% reduction in specific [³H]-NMS, was interpolated empirically between two concentrations of compound where percent inhibition was determined to be greater than and less than 50% respectively. The following table shows the pIC₅₀ figures for the Examples.

| Compound of | |
|---|---|
| Example No. | pIC₅₀ |
| 1 | 7.9 |
| 2 | 8.9 |
| 3 | 8.9 |
| 4 | 8.8 |
| 5 | 8.9 |
| 6 | 7.7 |
| 1f | 7.7 |
| 2a | 8.1 |
| 3c | 8.5 |
| 4a | 9.1 |

1. A compound of formula (I): 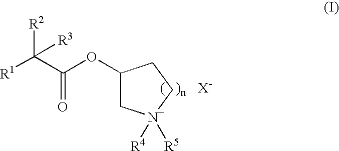
wherein:
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
R⁵ represents hydrogen or C₁₋₆ alkyl;
n is 1 or 2;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
and X represents a pharmaceutically acceptable anion of a mono or polyvalent acid.

2. A compound according to claim 1, wherein R¹ represents phenyl, which phenyl may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) and N(C₁₋₄ alkyl)₂.

3. A compound according to claim 1, wherein R² represents cyclopentyl which may be optionally substituted by one or more substituents independently selected from halogen, hydroxyl, C₁₋₆ alkoxy, NH₂, NH(C₁₋₄ alkyl), N(C₁₋₄ alkyl)₂ and C₁₋₆ alkyl, which C₁₋₆ alkyl may be optionally substituted by one or more substituents independently selected from halogen, hydroxyl, NH₂, NH(C₁₋₄ alkyl) nlld N(C₁₋₄ alkyl)₂.

4. A compound according to claim 1, wherein R³ represents methyl.

5. A compound according to claim 1, wherein R⁴ and R⁵ each independently represent methyl or ethyl.

6. A compound according to claim 1, wherein n is 1.

7. A compound according to claim 1, wherein n is 2.

8. A compound according to claim 1, wherein X represents bromide or iodide.

9. A compound according to claim 1, selected from:
(3S)-3-{[(2R)-2-Cyclopentyl-2-phenylpropanoyl]oxy}-1,1-dimethylpyrrolidinium X,
(3R)-3-{[(2R)-2-Cyclopentyl-2-phenylpropanoyl]oxy}-1,1-dimethylpyrrolidinium X,
(3S)-3-{[(2S)-2-Cyclopentyl-2-phenylpropanoyl]oxy}-1,1-dimethylpyrrolidinium X,
(3R)-3-{[(2S)-2-Cyclopentyl-2-phenylpropanoyl]oxy}-1,1-dimethylpyrrolidinium X,
(3R)-3-{[(2S)-2-Cyclopentyl-2-phenylpropanoyl]oxy}-1,1-dimethylpiperidinium X, or
(3S)-3-{[(2S)-2-Cyclopentyl-2-phenylpropanoyl]oxy}-1,1-dimethylpiperidinium X, wherein X represents a pharmaceutically acceptable anion of a mono or polyvalent acid

10. A process for preparing a compound of formula (I) as defined in claim 1, which comprises reacting a compound of formula (IV) wherein R¹, R² and R³ are as defined in formula (I) 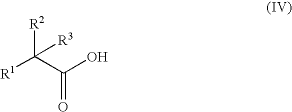
or a C₁₋₆alkyl ester, acid anhydride or acid halide thereof, with a compound of formula (V), wherein R⁴ and n are as defined in formula (I) 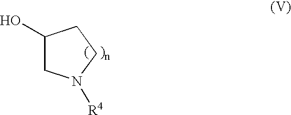
to yield a compound of formula (II) 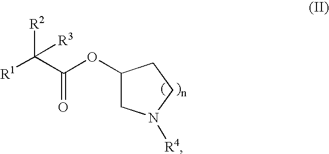
and subsequently reacting (II) with a compound of formula R⁵—Y (III), wherein Y is a leaving group and R⁵ is as defined in formula (I),
and optionally carrying out one or more of the following:
converting the compound to a further compound of formula (I),
forming a pharmaceutically acceptable salt with an anion of a mono or polyvalent acid.

11. A pharmaceutical composition comprising a compound of formula (I) as defined in claim 1, in association with a pharmaceutically acceptable adjuvant, diluent or carrier.

12. A process for the preparation of a pharmaceutical comprising a compound of formula (I) as defined in claim 1, in association with a pharmaceutically acceptable adjuvant, diluent or carrier, which comprises mixing a compound of formula (I), as defined in claim 1, with a pharmaceutically acceptable adjuvant, diluent or carrier.

13-14. (canceled)

15. A method of treating chronic obstructive pulmonary disease in a warm-blooded animal, which comprises administering to a mammal in need of such treatment an effective amount of a compound of formula (I) as defined in claim 1.

16. A pharmaceutical product comprising, in combination, a first active ingredient which is a compound of formula (I) as defined in claim 1, and a second active ingredient which is selected from;
a PDE4 inhibitor;
a selective β₂. adrenoceptor agonist;
a modulator of chemokine receptor function;
an inhibitor of p38 kinase function;
a glucocorticoid; and
a non-steroidal glucocorticoid receptor antagonist.

17. A compound of formula (II) 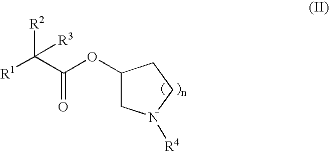
wherein,
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁴ represents hydrogen or C₁₋₆ alkyl;
n is 1 or 2;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl.

18. An optically pure compound of formula (IV), 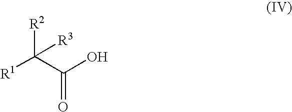
wherein,
R¹ represents phenyl, benzimidazolyl, benzthiazolyl, benzoxazolyl or a 5-6 membered heteroaromatic ring, each of which may be optionally substituted by one or more substituents independently selected from halogen, S(O)₀₋₂R⁶, NR⁷R⁸, S(O)₂NR⁹R¹⁰, C(O)NR¹¹R¹², C(O)₂R¹³, NR¹⁴S(O)₂R¹⁵, NR¹⁶C(O)R¹⁷, NR¹⁸C(O)₂R¹⁹, NR²⁰C(O)NR²¹R²², OR²³ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R² represents a C₃₋₈ cycloalkyl ring, which cycloalkyl ring may be optionally substituted by one or more substituents independently selected from halogen, S(O)₀₋₂R²⁴, NR²⁵R²⁶, S(O)₂NR²⁷R²⁸, C(O)NR²⁹R³⁰, NR³¹S(O)₂R³², NR³³C(O)R³⁴, OR³⁵ and C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R³ represents C₁₋₆ alkyl;
R⁶, R¹³, R¹⁵, R¹⁷, R¹⁹, R²³, R²⁴, R³², R³⁴ and R³⁵ each independently represent hydrogen or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alkyl)₂;
R⁷, R⁸, R⁹, R¹⁰, R¹¹, R¹², R¹⁴, R¹⁶, R¹⁸, R²⁰, R²¹, R²², R²⁵, R²⁶, R²⁷, R²⁸, R²⁹, R³⁰, R³¹ and R³³ each independently represent hydrogen, C₂₋₆ hydroxyalkyl or C₁₋₆ alkyl, which C₁₋₆ alkyl may be optionally substituted by one or more substituents independently selected from halogen, hydroxyl, C₁₋₆ alkoxy, NH₂, NH(C₁₋₆ alkyl) and N(C₁₋₆ alyl)₂;
or any of R⁷ and R⁸, R⁹ and R¹⁰, R¹¹ and R¹², R²¹ and R²², R²⁵ and R²⁶, R²⁷ and R²⁸, or R²⁹ and R³⁰, together with the nitrogen atom to which they are both attached, may form a 4-8 membered aliphatic heterocyclic ring, which heterocyclic ring may be optionally substituted by one or more substituents independently selected from halogen, hydroxyl, C₁₋₆ alkyl, C₁₋₆ hydroxyalkyl and C₁₋₆ haloalkyl;
or a C₁₋₆ alkyl ester, acid anhydride or acid halide thereof.
